(19) United States
(12) Patent Application Publication
(10) Pub. No.: US 2015/0216422 A1
(43) Pub. Date: Aug. 6, 2015

(54) Whole Body Infrared Thermography Systems And Methods

(71) Applicant: Daniel Beilin, Aptos, CA (US)

(72) Inventor: Daniel Beilin, Aptos, CA (US)

(21) Appl. No.: 14/173,875

(22) Filed: Feb. 6, 2014

(51) Int. Cl. A61B 5/01; G06F 19/00; A61B 5/00

(52) U.S. Cl. CPC A61B 5/015; A61B 5/0064; G06F 19/3487; G06F 19/366

(57) ABSTRACT

Disclosed herein is a method where a thermography device collects raw data that is forwarded to a server wherein the server runs the raw data through a database of signature parameters and generates a report. After the report is generated, the server notifies a technician that a report has been created and makes the report available online for the technician to login and retrieve. The separation of the measurement system and the analysis system allows some protection of trade secrets which are contained within the analysis software.

### CLAIM FOR PRIORITY

This application is a continuation-in-part of currently pending U.S. patent application Ser. No. 12/028,743 by the inventor of the same name and entitled, “Whole Body Infrared Thermography Systems and Methods,” and is incorporated by reference in its entirety and for all purposes to the same extent as if the patent application was specifically and reprinted here.

### FIELD OF THE INVENTION

The present invention relates to the field of thermographic diagnosis. The present invention, more particularly, relates to the collection, transfer, and relation of information collected from a thermography device.

### BACKGROUND OF THE INVENTION

Thermography is used for measuring the amount of body heat delivered to the skin from a combination of a person's cellular metabolism and their nervous system in targeted areas of the body. These measurements may be taken from a number of sites on the body. Each site projects body heat for particular internal organs. The amount of heat projected to the skin as well as the character of heat change after stimulus represents the condition of that region or organ. When conducting thermographic diagnosis, an operator will take a first measurement of temperature data at selected sites then subject the patient to some stimulus. After the stimulus, temperature data is from the selected sites is taken a second time. Comparison of the first and second measurements at selected sites reveals the reaction of various organs to stimulus. That reaction is determinative to the diagnosis of various conditions.

Presently, many conditions which can be diagnosed by this method are well known and have been conducted by professionals regularly. These known conditions include heavy metal poisoning, cranial structural imbalance, hypothyroid conditions, sinus blockage, auxiliary lymph issues, food intolerance, and many more. Thermographic diagnosis is generally very non-invasive, low-cost, and can be accomplished in a relatively short period of time. Therefore, using a thermograph to diagnose conditions is preferable when possible. Requiring a technician to review the data might be further time consuming, thus it would be advantageous to create a system which would automatically analyze the data given a set of parameters used to do so.

Of additional note, the list of known diagnoses with a thermograph is limited. The thermograph is a dynamic device which has many undiscovered uses. Technicians who discover conditions which may be diagnosed by thermograph may not wish to disclose their method of diagnosis.

Accordingly, there is a need for a system which allows diagnosis and automated diagnosis which is protected from disclosure.

### SUMMARY OF THE INVENTION

According to a first aspect of the present invention, a thermography device collects raw data which is forwarded to a server wherein the server runs the raw data through a database of signature parameters and generates a report. After the report is generated, the server notifies a technician that a report has been created and makes the report available online for the technician to login and retrieve. The separation of the measurement system and the analysis system allows some protection of trade secrets which are contained within the analysis software.

### BRIEF DESCRIPTION OF THE DRAWING

For a further understanding of the objects and advantages of the present invention, reference should be had to the following detailed description, taken in conjunction with the accompanying drawing, in which like parts are given like reference numerals and wherein:

FIG. 1 illustrates the use of a temperature probe according to certain aspects of the invention;

FIG. 2 is a simplified system diagram according to certain aspects of the invention;

FIGS. 3a-3c depict exemplary embodiments of a probe; and

FIGS. 4 & 5 are block diagrams illustrating functional elements found in exemplary embodiments of the invention.

FIG. 6 is a flowchart showing the method of patient data processing.

FIG. 7 is a sample display of data collection assistance.

### DETAILED DESCRIPTION OF THE INVENTION

Embodiments of the present invention will now be described in detail with reference to the drawings, which are provided as illustrative examples so as to enable those skilled in the art to practice the invention. Notably, the figures and examples below are not meant to limit the scope of the present invention to a single embodiment, but other embodiments are possible by way of interchange of some or all of the described or illustrated elements. Wherever convenient, the same reference numbers will be used throughout the drawings to refer to same or like parts. Where certain elements of these embodiments can be partially or fully implemented using known components, only those portions of such known components that are necessary for an understanding of the present invention will be described, and detailed descriptions of other portions of such known components will be omitted so as not to obscure the invention. In the present specification, an embodiment showing a singular component should not be considered limiting; rather, the invention is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the components referred to herein by way of illustration.

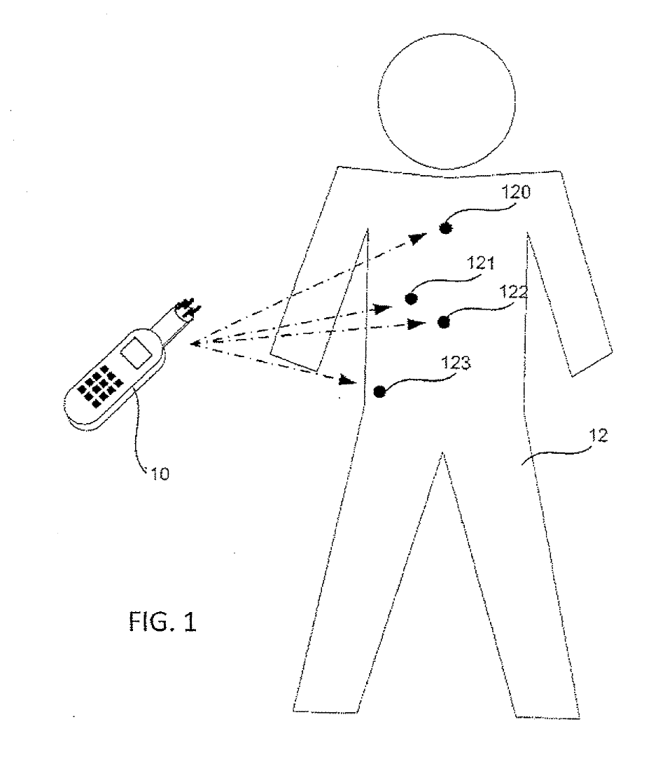

Certain embodiments of the invention provide systems and methods applicable in the practice of whole body thermography. With reference to FIG. 1, an infrared thermography device 10 can be used in the analysis of sequential changes in skin temperature at selected predetermined test sites 120-123 on a body 12. The predetermined test sites 120-123 can number in excess of 100 sites and typically exceed 120 test sites for a complete scan. In certain embodiments, the measured changes in skin temperature may be induced by stimuli that include cooling of ambient temperature, application of a noxious frequency and other stress stimuli. Patterns in the temperature changes at the predetermined test sites 120-123 can then be used for diagnosis of underlying medical conditions.

Certain embodiments support analyses of thermodynamic temperatures and changes in thermodynamic temperatures. Thermodynamic temperature is independent of the nature of a thermometric substance and the properties associated with a particular substance and is formulated in terms of the position of a fixed reference point and the size of the base unit. In the international standard temperature scale, the base unit is the fraction 1/273.16 of the thermodynamic temperature of the triple point of water, and the fixed reference point (zero Kelvin) is absolute zero. The base unit is 1 Kelvin (1K). Temperature is also measured in degrees Celsius (° C.). In the Celsius scale the base unit is the same as in the Kelvin scale, but absolute zero is at −273.16° C.

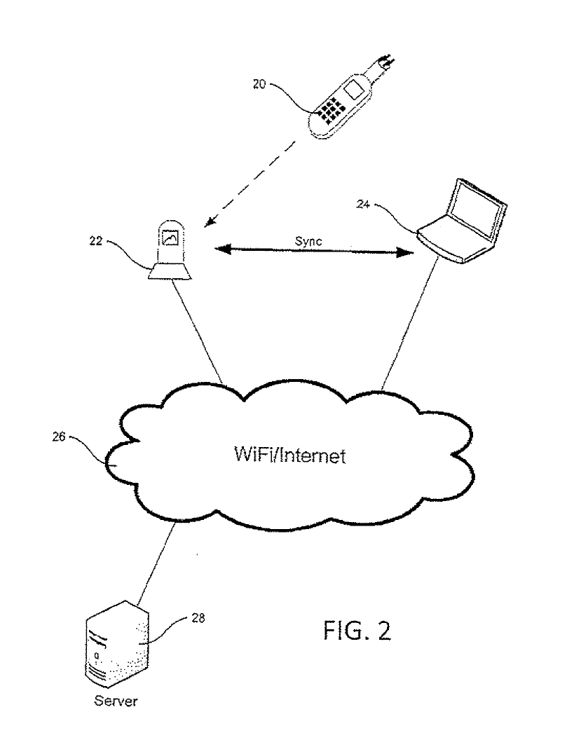

With reference to FIG. 2 certain embodiments of the invention comprise a base station 22 that supports operation of a temperature measuring probe 20. Probe 20 is typically operated by an operator or user trained to obtain temperature readings from predetermined test sites on a subject body. Base station 22 may provide a plurality of support services and functions for probe 20 and for the operator or user. In certain embodiments, base station 22 can receive temperature measurements obtained by probe 20 and can process the temperature readings before transmitting the processed results to a computing system 24. Probe 20 typically includes some processing capability as will be described in more detail below. Base station 22 may provide supplemental processing as well as data collection services to enable efficient communication between the probe 20, the base station 22, local computer 24 and one or more servers or data processing systems 26.

In certain embodiments, base station 22 and probe 20 may communicate using wired or wireless connections. In one example, data may be transferred from probe 20 using Bluetooth, WiFi or any suitable standard or proprietary wireless technology. In certain embodiments, probe 20 and base station 22 may be physically connected or physically connectable. In one example, a connecting cable may facilitate communication between probe 20 and base station 22. In another example, probe 20 may be docked with base station 22 enabling hard-wired communication between base station 22 and probe 20 and facilitating charging of batteries in probe 20 while the probe 20 is held in place by base station 22. Communication between base station 22 and probe 20 while docked or connected by cable may conform to a recognized commercial standard such as universal serial bus and/or to any other suitable standards-defined or proprietary communication protocol. While docked or connected by cable, probe 20 may exchange configuration information, software updates and measurement related data with the base station 22 and the local computer 24 or networked server 28. It will be appreciated that such information and data may just as well be exchanged wirelessly.

In certain embodiments, probe 20 and base station 22 may selectively communicate using a combination of wireless and wired communications methods where the communication method is selected based on factors including operator choice, location, ambient electromagnetic interference levels and regulations. In a medical facility, it may be necessary or required to curtail wireless communications in proximity to equipment that could be affected by wireless communications or that could create hostile environments for wireless communications. For example, communication between probe 20 and base station 22 may be restricted to wired communication where wireless communications used by the system might interfere with the operation of other patient monitoring equipment. In another example, proximity to electromagnetically noisy equipment may prevent secured reliable wireless communications between probe 20 and base station 22.

In certain embodiments, probe 20 and base station 22 may perform combinations of processes that enable a richer set of functionalities. For example, base station 22 may provide additional storage or buffers for data collected by probe 20. Furthermore, base station 22 may maintain configuration information and histories of prior readings obtained by probe 20. In certain embodiments, analysis of results may be performed by a combination of probe 20 and base station 22 although many embodiments provide a probe 20 that is capable of producing at least an initial analysis of results obtained by probe 20. In certain embodiments, probe may communicate directly with a local computer 24 or network server 28 and a base station 22 may be unnecessary for operation of the probe 20.

In certain embodiments, the primary functions of probe 20 include obtaining measurements of skin temperature of a subject at various test sites on subject's body. Measurements can be obtained as absolute temperature readings and may be expressed in degrees of Kelvin, Celsius or Fahrenheit as desired or selected by an operator. Measurements can also be obtained and expressed as a difference in temperature stated in degrees of Kelvin, Celsius or Fahrenheit. Difference readings may include one or more types of difference including a difference in successive readings at a single test site, a difference in readings between neighboring test sites, a difference between temperature at a test site and ambient temperature, a difference in measured temperature from a baseline temperature and so on. Probe 20 may record a combination of difference temperatures and absolute temperatures. In one example, probe 20 may record ambient temperature as an absolute reading and temperatures at a test site as variances from ambient temperature.

In certain embodiments, base station 22 receives temperature measurements obtained at a number of different test sites. Test sites may be grouped based on relationships that exist between group members and/or based on relevance of the group members to an analysis protocol and, potentially based on relevance of members to identification of one or more medical conditions. Groups may also comprise test sites that are to be measured in a desired sequence and/or timeframe. Temperature measurements may include a sequence of measurements obtained over a time period at a set of test sites. For example, the sequence may include two or more sets of readings, each set taken at different times. Analysis of the results may include a consideration of changes in conditions associated with the temperature measurements. For example, the subject from which measurements are taken may be located in a room in which ambient temperature is controlled to create stimuli that potentially affect skin temperature readings. In that regard, ambient temperature of the room may be adjusted after a first set of readings is obtained at a baseline ambient temperature. Adjustments to temperature may be made at a rate calculated to stimulate a desired response in the subject and, after a predetermined interval of time, a second set of readings may be obtained at the second ambient temperature. Analysis of results can include receiving information identifying the time and rate of change of ambient temperature. In certain embodiments, other stress stimuli may be applied that are calculated to obtain changes in body temperature. Application, nature and characteristics of a stimulus applied in this manner may be provided to the probe 20 for recording with associated measurements and analysis of results.

In certain embodiments, temperature measurements obtained from a plurality of test sites are analyzed by the probe 20 and, in at least some embodiments, by the base station 22. Typically, results are analyzed using pattern recognition techniques that may be embodied in computer programs and configured to identify one or more patterns in the collected results. Patterns may be detected within a selected set of test sites and may be further detectable between sets of measurements captured at different times. Consequently, patterns may be multidimensional in character having spatial and temporal components. In one example, temperature contours are mapped to a set of test sites and can then be used to identify temperature gradients on the skin of a subject. Additional sets of measurements can be obtained after a selected delay, and differences in the contours obtained from the successive sets of measurements can be mapped between points in time. As noted above, the differences may be induced by introduction of a stimulus to the subject between capture of the sets of measurements.

Pattern recognition techniques can be used to identify correlation between sets of results and reference patterns. The reference patterns may represent normal and/or ideal states. In certain embodiments, some reference patterns may represent abnormal states. Pattern matching may also be used to detect deviations from reference states. Thus, it may be possible to identify the onset of a medical condition or absence of medical condition by observing differences between measured results and results representing an ideal state. These differences may be monitored over a series of thermographic scans to identify trends away from normalcy and, in some instances, toward a known abnormal state. Additionally, it may be possible to detect trends towards normalcy when a subject had previously been associated with an abnormal or atypical medical condition.

In certain embodiments, patterns are identified using pattern recognition techniques that compare newly acquired sets of measured temperatures to models comprising patterns associated with certain medical conditions. Model patterns relevant to the current subject can be stored on the probe prior to measurement to facilitate real-time analysis. Testing may involve only a subset of the possible test sites of a subject. For example, the number of potential test sites may exceed 120 but certain test protocols may specify that only a portion of the test sites need be monitored during testing for a particular medical condition. A full scan of all test sites can be performed at any point in the test procedure to provide a spatial baseline of the subject, whereas other scans may be limited to one or more groups of test sites or region of the subject's body. Furthermore, the number of different groups and their constituents can be altered progressively or otherwise as a test protocol progresses.

In certain embodiments, the probe 20 may access additional model patterns available through the base station 22 and/or maintained by a local computer 24 or server 28 accessible through network 26. Model patterns may include a set of normal patterns representative of healthy subjects and deviations from the normal patterns that are indicative of a condition of interest. Patterns maintained by base station 22 or server 28 may include patterns associated with disease, deficiency and dysfunctions. For example, certain patterns of temperatures or temperature variations may be associated with diseases such as diabetes and cancers while other patterns may correspond with enzyme deficiencies and/or cognitive disorders. More than one pattern may be detected in a set of measurements and certain embodiments can correlate conditions with the presence of a plurality of detected patterns.

In certain embodiments, results obtained for a subject may be stored in the probe 20 for future comparison. Typically, results are also transferred to the base station and to a host computer 24 and/or server 28. Results intended for storage may be communicated using wireless or wired communications and may include transmitting the results over a network 26 such as the Internet. Results that are stored may include raw data comprising temperature measurements obtained by probe 20, ambient conditions including temperature, rate of change of temperature, air pressure, humidity, etc. and characteristics of stimuli applied to the subject. Raw data may be further processed by a central system using the same or different pattern recognition tools. Furthermore, additional analysis of the raw data may be performed if new algorithms and/or reference patterns become available. Raw data and analytical results may be used to augment or modify a model or other reference patterns. In one example, the stored information of a subject who develops an infirmity or medical condition subsequent to testing may be evaluated with other the information of other patients in order to develop new or different reference patterns.

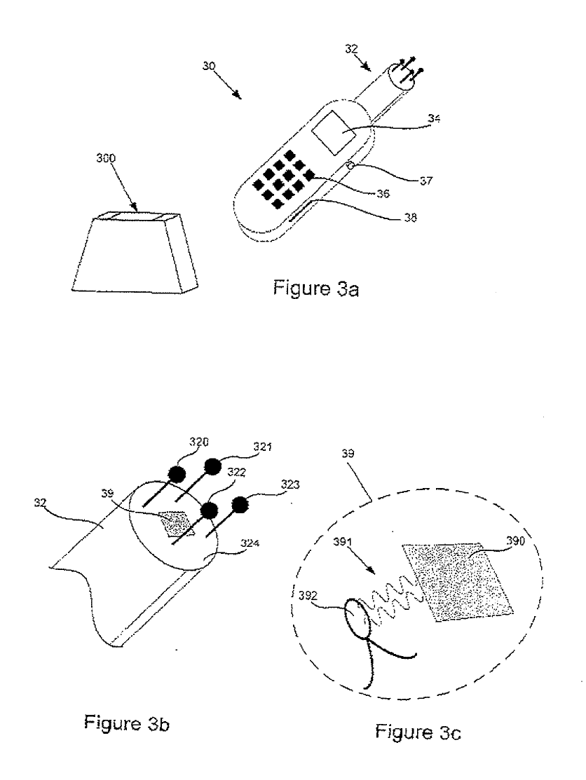

Referring now to FIG. 3a, a probe 30 may include an embedded computing system comprising a processor (not shown), a display 34, a communications interface provided internally or as a plug-in 38, one or more user input device 36, 37 and instructions for performing one or more pattern recognition algorithms. The processor may include one or more processing devices such as microprocessors, digital signal processors (“DSP”), custom logic arrays or other microcontroller. Communications interface 38 may be provided internal to probe 30 or may be provided as a plug-in component. A display 34 may be provided to provide system status, results including graphical displays and other information to an operator of the system. Input devices can include a keyboard/keypad 36, pushbutton 37, an optical reader, a microphone, etc. Typically, an operating system, measurement module, analysis tools and communications manager are provided to control temperature measurement and analysis of results. In certain embodiments, a real-time operating system is employed to provide accurate timing for measurement purposes.

In certain embodiments, a probe can be provided with instructions for conducting a testing procedure or protocol. For example, testing sites may be identified based on factors that include patient history and one or more targeted conditions. Thus, medical conditions associated uniquely with male patients may indicate a test procedure different from procedures for female patients. Similarly, certain medical conditions may have limited or no correlation with measurements observable at certain test sites. In certain embodiments, the test procedure can be conveyed to a user of the probe by means of an integral display. In some embodiments, an operator is prompted to obtain measurements from a next test site through a combination of textual description graphics and other signals. Locations for testing may be described textually in terms that describe a distance and direction (i.e. a vector) from known anatomical landmarks. In certain embodiments, an image identifying the site and/or its relationship to one or more anatomical landmark may be presented. In at least some embodiments, location information is provided audibly, typically through a wireless earphone or headset.

In certain embodiments, upon locating the test site, an operator can capture a temperature by indicating that the probe is positioned for a reading in a manner discussed below in more detail. Upon measuring temperature at the site, the probe typically provides a signal to move to the next test site. The signal may be audible and/or visual and may include information identifying the next test site for measurement. In certain embodiments, the probe may present the operator with an option to navigate backwards to a previous test site or skip the current test site. In that regard, the probe may also identify questionable measurements that should be retaken or verified. Measurements may be questionable if they fall outside a reasonable range, are identified as problematic by an operator or are otherwise inconsistent with expected results. In a simple example, a reading of 80° F. or 108° F. may be considered questionable. The range of expected or valid temperatures can be configured based on prior results and/or measurements taken at neighboring test sites.

Typically probe 30 is in wireless communication with a base station 300 or a computing system (not shown). Base station 300 can be provided to charge batteries of the probe and to support processors for manipulating data obtained by the probe 30. For example, in certain applications, it may be desirable to minimize power consumption of the probe 30 while providing rapid processing of measurements obtained by probe 30. In some embodiments, measurement obtained by probe 30 can be relayed at the first opportunity to a base station 300 for processing and results of the processing can be returned by the base station for display on the probe. In the event that certain measurements appear to be outside of certain ranges, the base station 300 may signal an operator to take repeat measurements at one or more sites. Signaling can be accomplished using the display and other output capabilities of the probe 30, such as audible signals including synthetic and/or prerecorded spoken instructions.

In certain embodiments, probe 30 is provided with sufficient storage and processing power to process measurements without communicating with the base station 300. For example, probe 30 may be preloaded with past histories of measurements taken from a subject to be measured. In addition, the probe 30 may maintain sufficient information in storage to permit recognition of patterns in measurements acquired from a scan of a subject. For example, a probe 30 may support sufficient memory to maintain patterns for plural medical conditions as well as instructions and parameters that cause one or more processors in the probe to perform a variety of pattern matching techniques on measured data. Information stored in the probe 30 may include information that guides processing on measurements in a general case as well as specific selections and sequences of processes to be performed for the individual subject, a group of subjects and/or a class of subject. For example, a group of subjects may include individuals identified as having indicators indicating elevated risk factors associated with a particular medical condition. A class of subjects may include male and female classes, pediatric and adult classes, etc. In certain embodiments, probe 30 maintains raw data and processed results in storage. Storage may be provided internally and/or on removable media such as a memory card connected through a card slot 38.

Certain aspects of probe 30 will now be described with particular reference to FIGS. 3b and 3c. In certain embodiments of the invention, probe 30 measures heat using one or more infrared sensors 39. In the depicted example, an infrared sensor 39 is arranged on a surface 324 on the end of column 32 of probe 30. Members or probe tips 320-323 are provided on surface 324 and may perform various roles. For example, probe tips 320-323 may be formed as rods having ends located at a desired distance from surface 324 in order to obtain a desired separation of sensor from surface to be measured. Providing a consistent separation can improve accuracy and repeatability of measurement. Furthermore, the probe tips 320-323 may be wired to a capacitive sensing circuit within the probe to trigger and end sampling of each point. In some embodiments, ridges, undulations and other textures may be provided on surface 324 to increase available surface area for deploying an infrared sensor.

In certain embodiments, and as shown in FIG. 3c, infrared sensor 39 can comprise heat transducers 392 and filters 390. Suitable heat transducers 392 can include thermocouples, thermistors, charge coupled devices, photovoltaic devices sensitive to infrared wavelengths, infrared imagers and so on. In one example a thermocouple 392 is mounted in a cavity. Cavity can be lined with heat absorbing material such that the thermocouple 392 can measure heat induced in the lining. Cavity can be parabolic in shape and line with a reflective material such that a thermocouple 392 or other heat transducer may be located at a focus or at a focal plane of the thus formed parabolic mirror such that the heat transducer 392 receives a substantial majority of the heat energy 391 incident on the sensor 39 and passing through filter 390.

Filters 390 are employed in certain embodiments of the invention to restrict temperature measurement to measurement of heat energy 391 found at certain electromagnetic wavelengths. Typically, heat energy detected as infrared wavelengths in the 2-20 μm range provides information useful in certain thermographical applications. In particular, the 2-20 μm range of wavelengths excludes heat energy that is absorbed (and re-radiated) by water. Consequently, filtering infrared energy incident on the sensor 39 can produce information that may be attributed to activities and conditions of tissues, organs and systems of the subject body. Different applications may be better supported with filters that have narrower bandwidths. In one example, infrared wavelengths in range 2-14 μm are measured. In another example, an 8-12 μm filter can produce measurements that are of particular interest in regard to certain medical conditions. In certain embodiments, filter 390 comprises a Germanium crystal. Other filters may also be used. As necessary, two or more filters may be used to obtain a desired response to incident light. For example, in a two filter system, one filter may exclude light other than infrared wavelengths 8-20 μm while the second filter may pass only wavelengths in the band 2-12 μm; the resultant filter passes light in the 8-12 μm band.

In certain embodiments, ends of probe tips 320-323 can be electrically conductive such that probe 30 may include a voltage or current source that, when applied to the skin through the ends of probe tips 320-323 can be used to determine a measurement of electrical conductivity of the skin. Thus, resistances and impedances may be measured between combinations of pins, including between pairs of pins and between one pin and a plurality of other pins. Characteristics of currents passed through the skin may be used to determine resistance, inductance and capacitance.

In certain embodiments, the detection of skin impedance can be used to enable or start a temperature reading. In some embodiments, adequate skin contact is determined when measured impedance of the probe tips 320-323 falls within a range consistent with skin impedance. Contact with the skin can then be automatically determined and a temperature reading acquired without operator intervention; a signal can then be sent to the operator to move to the next test site. The probe display 34 may identify the next site to be tested enabling rapid navigation and temperature measurement acquisition from identified test sites.

In certain embodiments, one or more of pins 320-323 may be configured to mechanically activate/deactivate corresponding switches upon contact with the skin. Thus, when the switch is activated, the measurement of temperature can be enabled or initiated. More than one switch may be employed and multiple switches may be used to ensure proper alignment of the probe with the skin surface. Additionally, proximity detection may be performed by the probe 30 using non-physical means. For example, an angled spot of light may be projected on the skin by an LED and detected by a suitably positioned detector. In some embodiments, temperature is recorded only at the instruction of an operator. To this end, one or more push buttons 37 may be located on the probe 30 to allow the operator to command the probe 30.

In certain embodiments, and as illustrated in the example of FIG. 3a, the tips of pins 320-323 are provided with generally spherical, or ellipsoidal ends. In some embodiments, the tips may be pointed, flattened or shaped as desired or necessitated by the application and types of measurements to be obtained.

Certain embodiments of the invention can provide a cost-effective measurement system capable of temperature resolution that is better than 0.1° C. and rapid response times. In the example, depicted, the probe 30 may be consistently located at an optimum distance from the site to be measured. While the probe in the example is suited for point measurement of skin-surface temperature and for integrated measurement of small skin areas, other embodiments may provide a plurality of probes that can be contacted to plural test sites on the skin of a subject.

In certain embodiments, probe 30 includes an ambient temperature measurement capability. However, in many embodiments, the ambient temperature may be obtained from an external device that is less affected by body heat of the subject or operator of the system. Ambient temperature may be communicated to the probe using wireless communications facilities of the probe such that temperature measurements may be stored as a temperature pair including ambient and measured contact temperatures. Ambient temperatures may be measured using a temperature sensor identical to the sensor 39 of the probe 30 in order to accommodate non-linear characteristics of the devices used in the sensor.

In certain embodiments, temperature measurements are gathered as part of a dynamic temperature study. A baseline temperature profile of a subject is typically obtained at a first temperature. Typically, the subject has been exposed to the first temperature for a time sufficient to stabilize skin surface temperature when the baseline is obtained. One or more stimuli may then be applied to cause the skin temperature to change. For example, ambient temperature may be lowered and one or more sets of subsequent skin temperature measurements are obtained after time intervals, typically determined by the parameters of the study. In certain embodiments, a second or later set of readings is obtained after a predetermined time period that permits the establishment of steady state conditions. However, temperatures may continue to settle or regress to base line levels. Consequently, it may beneficial to obtain subsequent temperature measurements over a relatively short period of time. Additionally, some test protocols may require that temperatures be acquired from certain test sites within a predetermined time interval, while changes are occurring.

Therefore, timed test procedures may be supported by a probe 30. The probe 30 can be equipped with temperature sensors that quickly converge on an accurate measurement of temperature. Additionally, the sensors may be controlled such that a final temperature may be accurately predicted before convergence is completed. In one example, in certain dynamic studies where it is of particular importance that response time be as short as possible, a processor can be used to calculate the stable final temperature based on the rate of change and elapsed time of the measurement, permitting determination of the stable final temperature more rapidly than could be reached by the detector.

When a probe 30 is to be moved relatively quickly from site to site, the probe 30 may be configured to set maximum and/or minimum times between readings and can provide prompts and other signals to an operator accordingly. In certain embodiments, multi-sensor probes or probe systems may be used to capture a plurality of readings simultaneously. For example, a probe may be configured with multiple temperature sensors positioned and oriented to obtain a plurality of readings from sites arranged around a central point of interest. Specific readings can then be selected for analysis and, in at least some embodiments, temperatures at desired test sites may be approximated or interpolated from neighboring measured test sites.

In certain embodiments, the probe may be used in conjunction with a multiple site monitoring system. The latter system can typically provide temperature monitoring at predetermined test sites enabling real-time monitoring of the predetermined sites. Additionally, a hand-held probe can be used to capture measurements at additional sites. The additional readings can be adjusted based on trends detected at the predetermined sites for which real-time measurements are obtained.

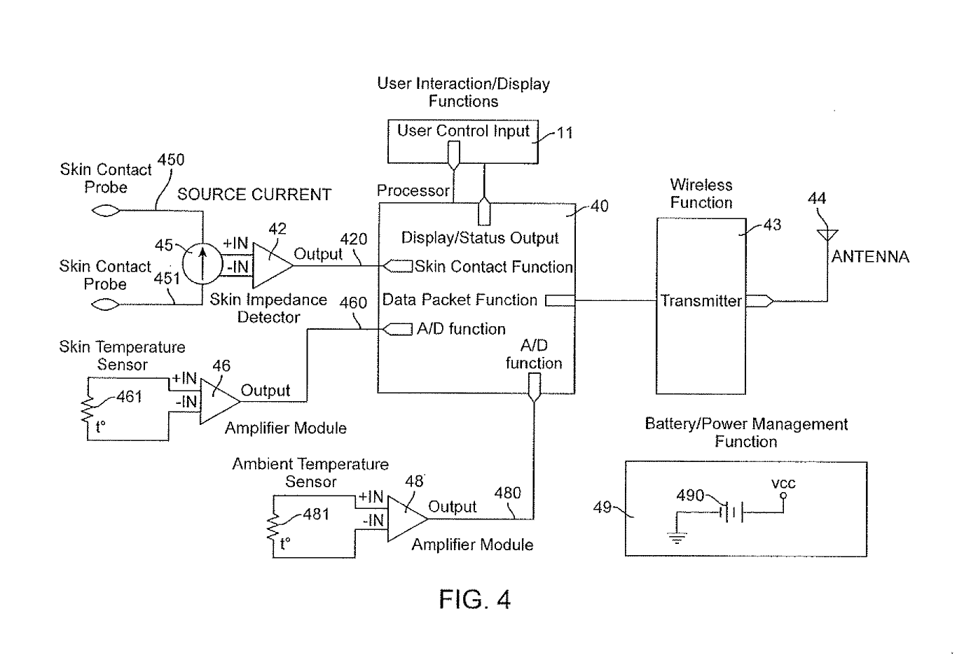

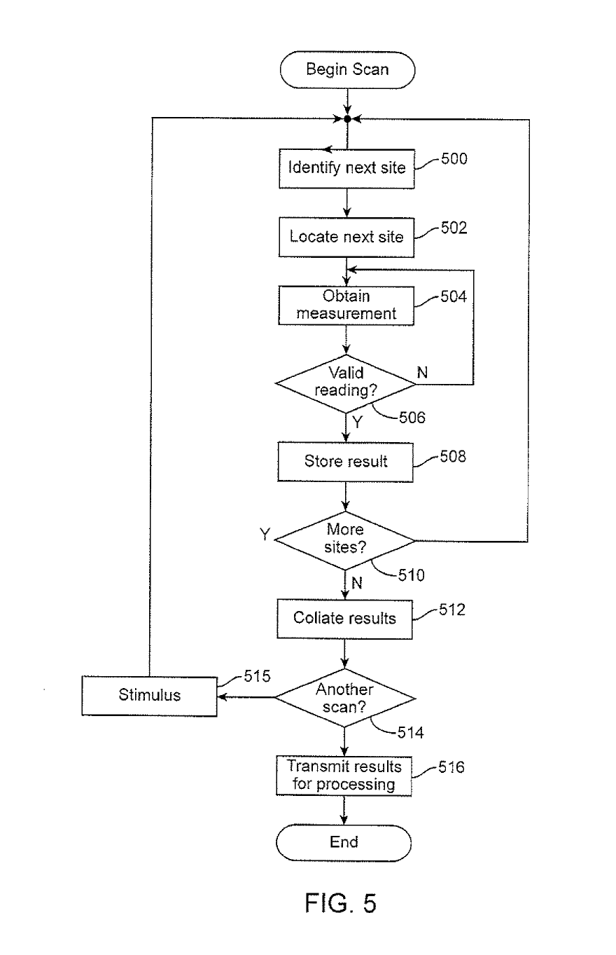

FIG. 4 includes a block diagram identifying elements of an example system provided according to certain aspects of the invention. Skin contact probes 450 and 451 provide a current from current source 45 to the subject skin. A comparator or voltage detector 42 measures voltage drop across current source 45 in order to provide a measurement 420 of skin impedance to processor 40. Skin temperature sensor 461 is monitored by voltage detector 46 which provides a signal 460 representing skin temperature to processor 40. Ambient temperature sensor 481 is monitored by voltage detector 48 which provides a signal 480 representing ambient temperature to processor 40. A user interface 11 is controlled by the processor 40, the user interface comprising switches, audio transducers including microphones, buzzers and loudspeakers, and display elements. Communications interface 43 facilitates communication between processor 40 and external devices, typically using wireless transmission through an antenna 44. Power management system 49 supports internal batteries 490 and external power supplies and controls charging of the batteries 490.

Turning now to FIG. 5, one example of a process for performing thermography is depicted. At step 500, a test site is identified at which a next temperature measurement should be taken. As described above, an operator maybe presented with a description or a graphic identifying the test site, typically in relation to an anatomical landmark. At step 502, the operator finds the test site and positions a probe adjacent to, or centered on the test site and at step 504, the temperature is measured. The temperature measurement may be triggered automatically based on location sensing devices on the probe and may also be indicated by activation of a button by the operator. When the probe has successfully obtained a temperature reading, the value may be checked for validity. To that end, the probe may maintain certain range information identifying maximum and minimum expected values of temperature. Validity may also be judged based on consistency of the reading with prior readings from the location or from neighboring locations. If the measurement is deemed invalid at step 506, a new measurement may be required, in which case the probe is typically removed from the subject skin and repositioned. In certain circumstances, the operator may be required to verify the location of the test site with regard to one or more anatomical landmarks.

When a valid temperature measurement has been obtained, the result is stored by the probe at step 508. The result may also be transmitted to a base station or workstation or network server. In certain embodiments, a display on the probe may present information derived from the measurement. For example, the value measured can be displayed along with other recent measurements at adjacent sites. In some instances, the result may be displayed together with previous or expected results for the site. If, at step 510, other sites are to be measured, steps 500-508 are repeated; otherwise, results may be collated at step 512. At step 512, results may be assembled into one or more sets of results that can be associated with points on the subject body, regions of the subject body and specific conditions. Collated sets of results can be stored locally as one of a series of scans and can be combined with other scans of the subject.

At step 514, it is determined whether the test protocol calls for another scan of the subject. Another scan may be performed for identical or different test points and may have some different and some common test points. Additionally, a next scan may be performed after introduction of a stimulus at step 515. Stimulus may include a heating or cooling of the ambient temperature. Depending on the nature of the stimulus and the type of analysis to be performed, a next scan may be performed after elapse of a predetermined time interval following the provision of the stimulus.

Results may be submitted for further processing at step 516. Processing may be performed for a complete set of results or by regions of the subject body. Results can be processed for a single test site or for a group of related test sites even if the sites in the group are not confined to a common region of the body. Results may be processed using previously obtained results. In certain embodiments, processing is performed locally within a combination of probe, base station and local computer. Additionally, in certain embodiments, further processing can be performed as a network service. In one example, results can be transferred to a network server for more detailed processing that may include advanced pattern recognition using a broader library of reference models and patterns. Network service may be provided to users on a subscription basis whereby a subscriber can request advanced or detailed processing according to agreed terms and conditions. Users may also submit results for processing subject to a per-use charge.

Certain embodiments of the invention provide a probe for measuring skin temperature, comprising an infrared sensor mounted at an end of the probe, a filter that restricts infrared radiation received by the infrared sensor to a selected band of Wavelengths, a processor for receiving from the sensor, a signal representative of a temperature at the skin surface and configured to process a plurality of temperatures measured at different test sites on the skin surface and a display providing information to an operator of the probe, the information including an analysis of the plurality of temperatures. In some of these embodiments, the filter comprises one or more Germanium crystals. In some of these embodiments, the filter is configurable to the selected band of Wavelengths. In some of these embodiments, the filter is configurable by adding or removing certain of the Germanium crystals. In some of these embodiments, the filter is configurable by replacing the filter with a different filter having different optical properties. In some of these embodiments, the band of Wavelengths comprises a portion of the Wavelengths in the range 2-20 um. In some of these embodiments, the band of Wavelengths comprises Wavelengths lying Within 4 pm band. In some of these embodiments, the band of Wavelengths comprises Wavelengths lying Within 2 um band. Some of these embodiments further comprise one or more elongated members extending a predetermined distance from the end of the probe for maintaining a desired separation of the infrared sensor and the skin surface.

Certain embodiments of the invention provide an infrared thermography method comprising the steps of identifying a target location on the skin of a subject, providing location information to an operator of a thermographic probe, the location information indicating the relationship of the target location to one or more landmarks, upon receiving a positioning signal, measuring temperature of the target location using an infrared sensor in the thermographic probe, wherein the positioning signal indicates a predefined proximity to the skin at the target location, and selectively repeating the identifying, providing and measuring steps for a plurality of different target locations. In some of these embodiments, the step of measuring includes obtaining a plurality of temperature readings from the infrared sensor over a selected period of time and determining a steady state temperature reading for the target location from the plurality of temperature readings. In some of these embodiments, determining the steady state temperature includes performing a statistical analysis of the plurality of temperature readings. In some of these embodiments, the step of measuring includes receiving infrared radiation from the target location, Wherein the infrared radiation is limited to a selected band of Wavelengths. In some of these embodiments, the band of Wavelengths is selected by one or more filters. In some of these embodiments, the one or more filters include at least one Germanium filter. In some of these embodiments, the step of measuring includes receiving infrared radiation from the target location, the infrared radiation being limited to a first band of Wavelengths. Some of these embodiments further comprise selecting a second band of Wavelengths and repeating the steps of identifying, providing and measuring for the second band of Wavelengths. In some of these embodiments, at least some of the Wavelengths are found in the first and second bands of Wavelengths. In some of these embodiments, the first and second bands Wavelengths comprise no common Wavelengths. In some of these embodiments, the second band of Wavelengths is narrower than the first band of Wavelengths. In some of these embodiments, identifying the target location includes selecting a next target location from a set of locations on the skin of the subject, the set of locations providing information related to the medical condition of the subject. In some of these embodiments, providing location information includes displaying an image depicting the target location in relation to the one or more landmarks. In some of these embodiments, providing location information includes providing an audible description of the target location in relation to the one or more landmarks. Some of these embodiments further comprise comprising communicating the measured temperature to a networked device.

Certain embodiments of the invention provide an infrared thermography diagnostic system comprising a thermographic probe providing a plurality of temperature measurements obtained at a plurality locations on the skin of a subject, each temperature measurement obtained using a band-limited infrared sensor, a processor configured to process the plurality of skin temperature measurements to determine the existence of one or more patterns and a repository of pattern information, each pattern identifying an underlying medical condition, wherein the plurality of temperature measurements includes a first series of temperatures measured at a first ambient temperature and a second series of temperatures measured at a second ambient temperature, each measurement in the first series and a corresponding measurement in the second series being obtained at the same location on the skin. In some of these embodiments, the processor receives the plurality of measurements from the thermo graphic probe and performs one or more pattern matching methods on the plurality of measurements using the repository of pattern information from a network server. In some of these embodiments, the one or more pattern matching methods is based on a trend analysis of the plurality of measurements.

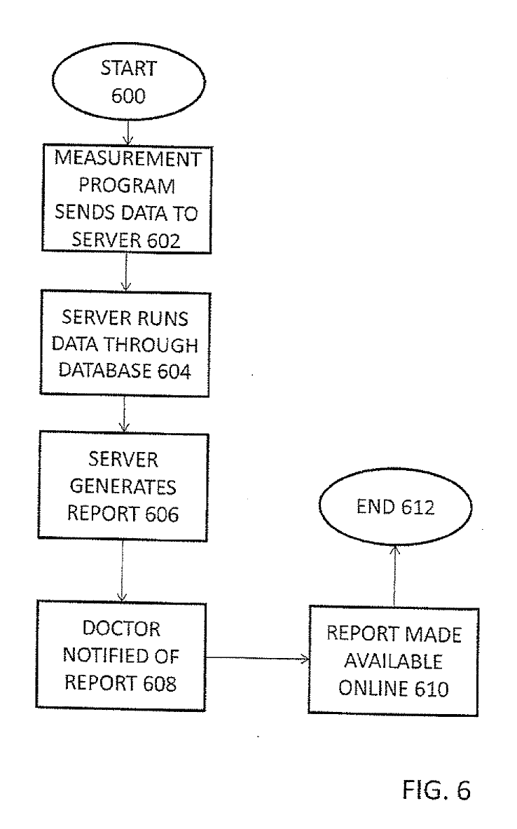

The invention will now be described with respect to FIG. 6, FIG. 6 is a flowchart showing the method of patient data processing. This flowchart begins assuming data has been collected from a thermographic device. This device may be thermography device 10 (of FIG. 1) or some other suitable device known in the art. Once the data is accepted by the measurement device and associated program, that data is forwarded to a server (602). This server may be remote from the device upon which the measurements are initially taken and stored. The sending or transmission can be accomplished over the Internet, or any other acceptable network known in the art. It is feasible that there would be many data collection devices which would reside in a plurality of unrelated locations and comparatively fewer servers which would be located either all in the same place or placed strategically to minimize cost of operation of the necessary connection network. Once acquired, the server will subject the data collected to a database of signature patterns stored locally on the server (604). The database of signature patterns is an automatic way of interpreting the data and analyzing it for conclusions. The contents of the database consist of known data patterns, data patterns disclosed in other pending patent applications, or trade secrets. Once subjected to the signature patterns in the database, the server generates an automated report based on matching signatures in the received data and the database entries (606).

Once created, the doctor or technician who collected the raw patient data is notified that the report has been created by the server (608). This notification could come by email, phone, SMS or MMS message, or any other suitable rapid notification process known in the art. The report is then made available online by the server for the doctor or technician to retrieve (610). The report format could be a PDF, a document created with “Microsoft Office,” or any other suitable printable electronic presentation format known in the art. In order to be retrieved online, the server may require the doctor or technician to login to the server with an individualized account information and password. This login procedure would not allow the doctor or technician who collected the result to access the server in any other capacity, thereby keeping the database of signature patterns removed from those without administrative server access.

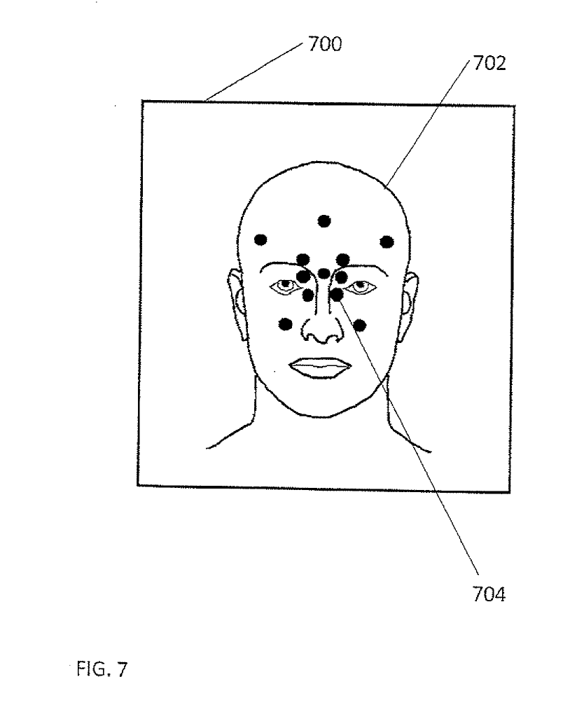

Referring now to FIG. 7, FIG. 7 is a sample display of data collection assistance. The program that operates the transfer and analysis of data discussed in FIG. 1 may also assist the collector of data in carrying out their duties. Using the computer 24 (of FIG. 2) as a display 700, use of the thermography device 10 (of. FIG. 1) can be directed. A body image 702 can be shown on the display 700 of the computer 24 which includes graphical depictions of corresponding locations 704 on the patient body where the thermography device 10 needs to collect patient data. The graphical depictions of corresponding locations can be highlighted on the display 700 as each is requires measurement by the thermography device 10.

What is claimed is:

1. A probe for measuring skin temperature, comprising:
an infrared sensor mounted at an end of the probe;
a filter that restricts infrared radiation received by the infrared sensor to a selected band of wavelengths;
a processor for receiving from the sensor, a signal representative of a temperature at the skin surface and configured to process a plurality of temperatures measured at different test sites on the skin surface; and
a display providing information to an operator of the probe, the information including an analysis of the plurality of temperatures.

2. The probe of claim 1, wherein the filter comprises one or more Germanium crystals.

3. The probe of claim 2, wherein the filter is configurable to the selected band of wavelengths.

4. The probe of claim 3, wherein the filter is configurable by adding or removing certain of the Germanium crystals.

5. The probe of claim 3, wherein the filter is configurable by replacing the filter with a different filter having different optical properties.

6. The probe of claim 1, wherein the band of wavelengths comprises a portion of the wavelengths in the range 2-20 μm.

7. The probe of claim 6, wherein the band of wavelengths comprises wavelengths lying within 4 μm band.

8. The probe of claim 6, wherein the band of wavelengths comprises wavelengths lying within 2 μm band.

9. The probe of claim 1 and further comprising one or more elongated members extending a predetermined distance from the end of the probe for maintaining a desired separation of the infrared sensor and the skin surface.

10. An infrared thermography method comprising the steps of:
identifying a target location on the skin of a subject;
providing location information to an operator of a thermographic probe, the location information indicating the relationship of the target location to one or more landmarks;
upon receiving a positioning signal, measuring temperature of the target location using an infrared sensor in the thermographic probe, wherein the positioning signal indicates a predefined proximity to the skin at the target location; and
selectively repeating the identifying, providing and measuring steps for a plurality of different target locations.

11. The method of claim 10, wherein the step of measuring includes:
obtaining a plurality of temperature readings from the infrared sensor over a selected period of time; and
determining a steady state temperature reading for the target location from the plurality of temperature readings.

12. The method of claim 11, wherein determining the steady state temperature includes performing a statistical analysis of the plurality of temperature readings.

13. The method of claim 10, wherein the step of measuring includes receiving infrared radiation from the target location, wherein the infrared radiation is limited to a selected band of wavelengths.

14. The method of claim 13, wherein the band of wavelengths is selected by one or more filters.

15. The method of claim 14, wherein the one or more filters includes at least one Germanium filter.

16. The method of claim 11 wherein the step of measuring includes receiving infrared radiation from the target location, the infrared radiation being limited to a first band of wavelengths, and further comprising:
selecting a second band of wavelengths; and
repeating the steps of identifying, providing and measuring for the second band of wavelengths.

17. The method of claim 16, wherein at least some of the wavelengths are found in the first and second bands of wavelengths.

18. The method of claim 16, wherein the first and second bands comprise no common wavelengths.

19. The method of claim 16, wherein the second band of wavelengths is narrower than the first band of wavelengths.

20. The method of claim 10, wherein identifying the target location includes selecting a next target location from a set of locations on the skin of the subject, the set of locations providing information related to the medical condition of the subject.

21. The method of claim 10, wherein providing location information includes displaying an image depicting the target location in relation to the one or more landmarks.

22. The method of claim 10, wherein providing location information includes providing an audible description of the target location in relation to the one or more landmarks.

23. The method of claim 10 and further comprising communicating the measured temperature to a networked device.

24. An infrared thermography diagnostic system comprising:
a thermographic probe providing a plurality of temperature measurements obtained at a plurality locations on the skin of a subject, each temperature measurement obtained using a band-limited infrared sensor;
a processor configured to process the plurality of skin temperature measurements to determine the existence of one or more patterns; and
a repository of pattern information, each pattern identifying an underlying medical condition, wherein the plurality of temperature measurements includes a first series of temperatures measured at a first ambient temperature and a second series of temperatures measured at a second ambient temperature, each measurement in the first series and a corresponding measurement in the second series being obtained at the same location on the skin.

25. The system of claim 24 wherein the processor receives the plurality of measurements from the thermographic probe and performs one or more pattern matching methods on the plurality of measurements using the repository of pattern information from a network server.

26. The system of claim 25 wherein the one or more pattern matching methods is based on a trend analysis of the plurality of measurements.
